(19) United States
(12) Patent Application Publication
(10) Pub. No.: US 2005/0267430 A1
(43) Pub. Date: Dec. 1, 2005

(54) Urine absorbent article

(76) Inventors: Shoko Maeda, Mitaka-shi (JP); Sakiko Maeda, Mitaka-shi (JP); Saeko Maeda, Mitaka-shi (JP)

Correspondence Address: Shoko Maeda, 2-21 Shimo-renjaku 9-chome, Mitaka-shi, Tokyo, 181-0013, JP

(21) Appl. No.: 10/855,181

(22) Filed: May 27, 2004

(51) Int. Cl. A61F 13/15

(52) U.S. Cl. 604/385.01; 604/1; 604/385.16

(57) ABSTRACT

The urine absorbent article includes a urine absorbent body, having an absorbent part, capable of absorbing bodily fluids and moisture; a connection part which is attached to an end portion of the urine absorbent body; and a stopper which is attached to the connection part, capable of preventing to slip or come off from the urine absorbent body when in use. Therefore, it can process sanitarily without increasing burdens of a care worker's labor and giving displeasure to other people and can throw away

### BACKGROUND OF THE INVENTION

The present invention relates generally to a urine absorbent article which absorbs bodily fluids such as urine and moisture.

After bedridden people or sick people etc. urinated in the conventional container currently generally called urine bottle, the nurse, etc. brings it to the toilet, discharges the bodily fluids and washes it, it is again used.

Therefore, since the conventional containers such as urine bottle is needed to work to discharge the urine and wash in the container, there are many labor burdens to the nurses and they have displeasure.

Moreover, while bringing to the toilet etc., the urine in a container is spilt and the floors of the room, passage etc. are soiled. When the container containing urine is left indoors, it is filled with a bad smell, it becomes stinking and is insanitary.

### SUMMARY OF THE INVENTION

Accordingly, it is an object to provide a urine absorbent article which can process sanitarily without increasing burdens of a care worker's labor and giving displeasure to other people and can throw away

The present invention is understood to encompass embodiments which include all or only a portion of the above objects, features and advantages which, unless recited in claims defining the invention, are understood not to limit interpretation of such claims. The above, and other objects, features and advantages of the present invention will become apparent from the following description read in conjunction with the accompanying drawings, in which like reference numerals designate the same elements.

It is to be expressly understood, however, that the drawings are for the purpose of illustration and description only, and are not intended as a definition of the limits of the invention.

### BRIEF DESCRIPTION OF THE DRAWINGS

FIG. 1 is a front view showing a first embodiment of the present invention;

FIG. 2 is a top view;

FIG. 3 is a back view;

FIG. 4 is a right side view;

FIG. 5 is a left side view;

FIG. 6 is a cross sectional view taken along the line 6-6 in FIG. 3;

FIG. 7 is a schematic explanation view of stopper;

FIG. 8 is a schematic explanation view when in use;

FIG. 9 is a side view showing a second embodiment of the present invention;

FIG. 10 is a cross sectional view taken along the line 10-10 in FIG. 8;

FIG. 11 is a front view showing a third embodiment of the present invention;

FIG. 12 is a bottom view;

FIG. 13 is a cross sectional view taken along the line 13-13 in FIG. 12;

FIG. 14 is a front view showing a fourth embodiment of the present invention;

FIG. 15 is a cross sectional view taken along the line 15-15 in FIG. 14;

FIG. 16 is a front view showing a fifth embodiment of the present invention;

FIG. 17 is a bottom view;

FIG. 18 is a side view;

FIG. 19 is a cross sectional view taken along the line 19-19 in FIG. 17;

FIG. 20 is a front view showing a sixth embodiment of the present invention; and

FIG. 21 is an extended view.

### DETAILED DESCRIPTION OF THE PREFERRED EMBODIMENTS

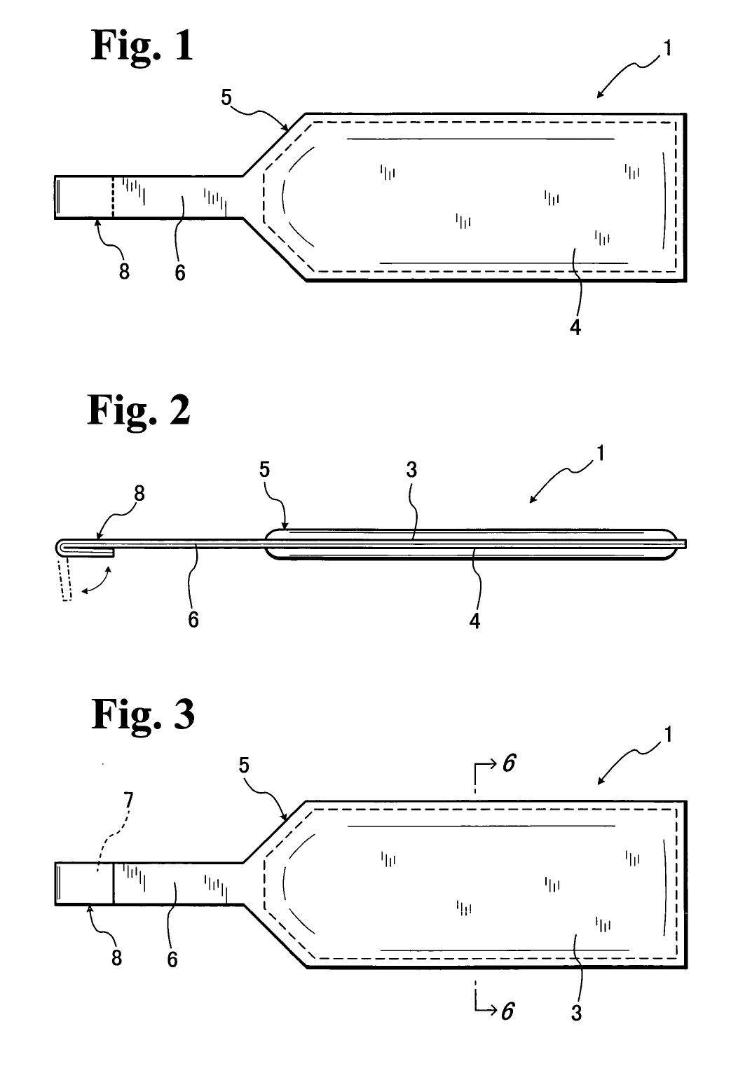

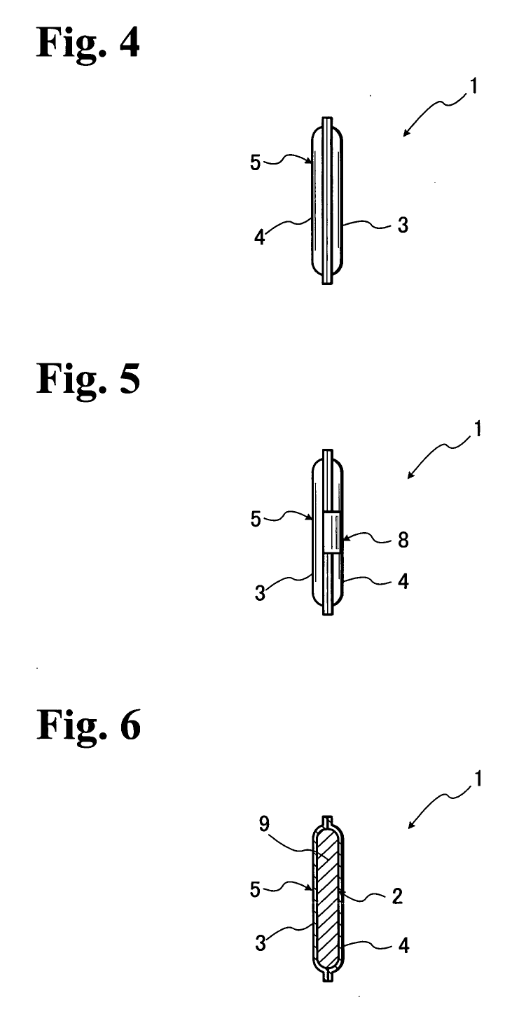

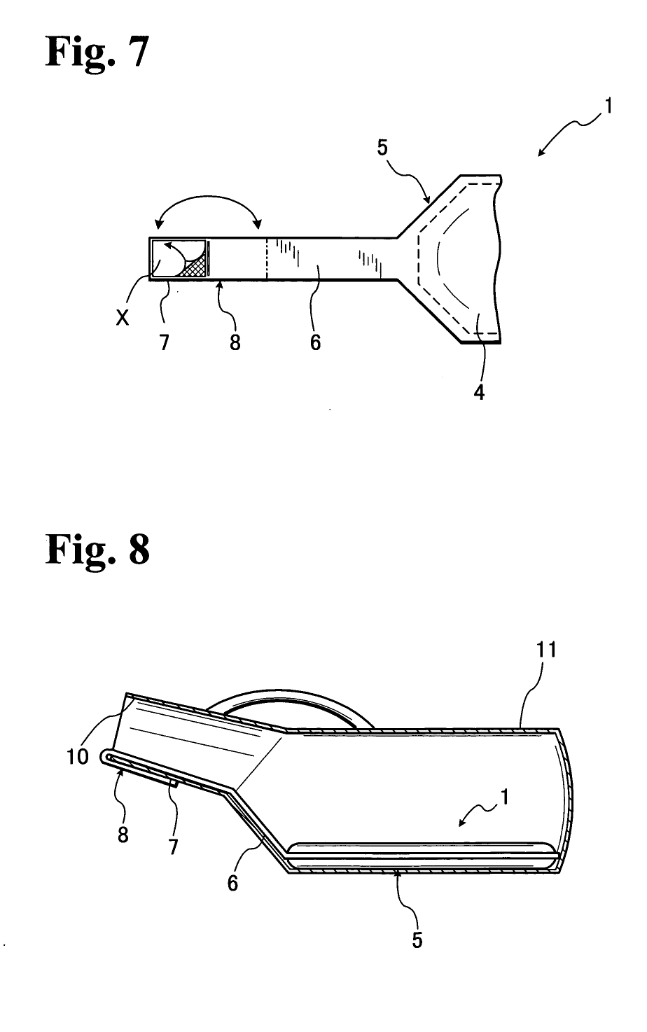

Preferred embodiments of the present invention are described in more detail below referring to the accompanying drawings. An understanding of the present invention may be best gained by reference FIGS. 1 to 8.

The numeral 1 shows a urine absorbent article of this invention, which absorbs bodily fluids such as urine and moisture and can be thrown away. The urine absorbent article is comprised of a urine absorbent body 5 formed shape of a square substantially, having an absorbent part 2, which can absorb bodily fluids such as urine and moisture, between a top sheet 3 and a back sheet 4 with anti-leak; a connection part 6, formed in the shape of a belt, which is attached to an end portion of the urine absorbent body 5; and a stopper 8 having an adhesion part 7, which is attached to an end portion of the connection part 6, capable of preventing to slip or come off the urine absorbent body 5 when in use.

The absorbent part 2 includes an absorbable sheet 9, such as cotton pulp currently used with the disposable diaper etc., absorbable paper and high polymer absorber material.

For the urine absorbent article 1, as shown in FIG. 8, after the urine absorbent body 5 inserts in a urine bottle 11 via an opening 10 inserting or attaching the predetermined parts of elderly people, sick person and the handicapped, an exfoliation paper X is removed. Then the adhesion part 7 of the stopper 8 is adhered fixedly to the outer circumferential part of the opening 10.

When in use, the urine bottle 11 is used by the same method as usual. When the urine is discharged, the sound of urination is muffled since it is absorbed by the absorbent part 2 of the urine absorbent body 5 quickly, and the urine does not stay in the urine bottle 11 since it is absorbed by absorbent part 2.

When the urine absorbent article 1 which absorbed urine is disposed, the adhesion part 7 is tore off from the opening 10, and the urine absorbent body 5 with the absorbed urine is taken out and disposed.

Therefore, since the urine is not spilt when the urine absorbent article 1 is carried or dropped, it can be dealt with sanitarily without leaking and falling urine.

Moreover, when it is putting in the room for a long time, and indoor environment can be kept good without filling with bad smell.

In addition, the urine bottle 11 for males is explained in this embodiment, and the urine bottles for women, for children, etc. may be used in this invention.

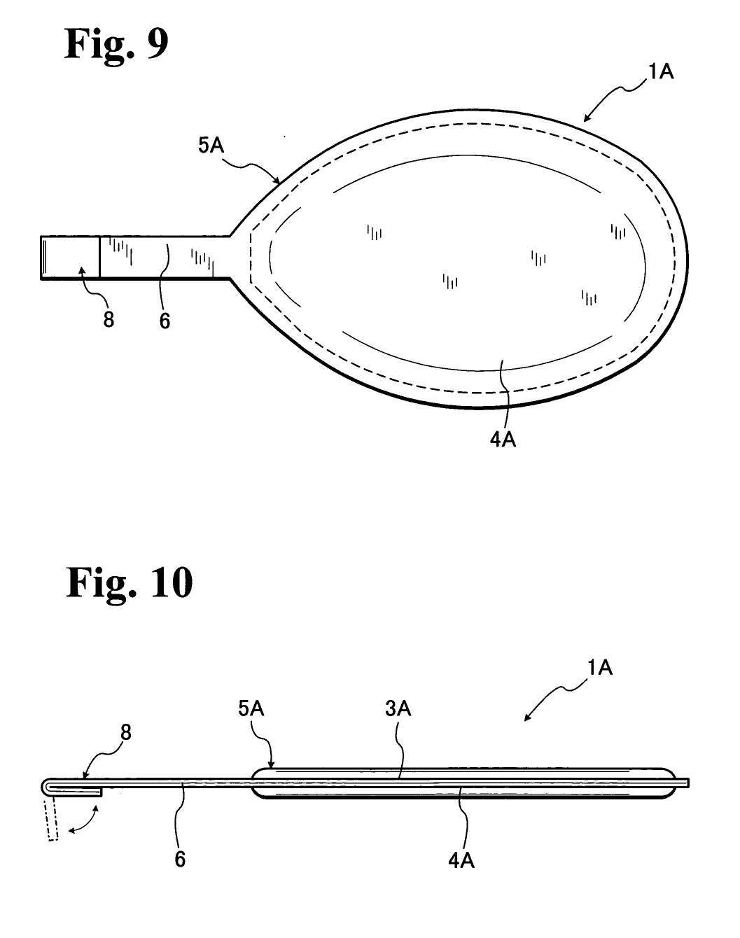

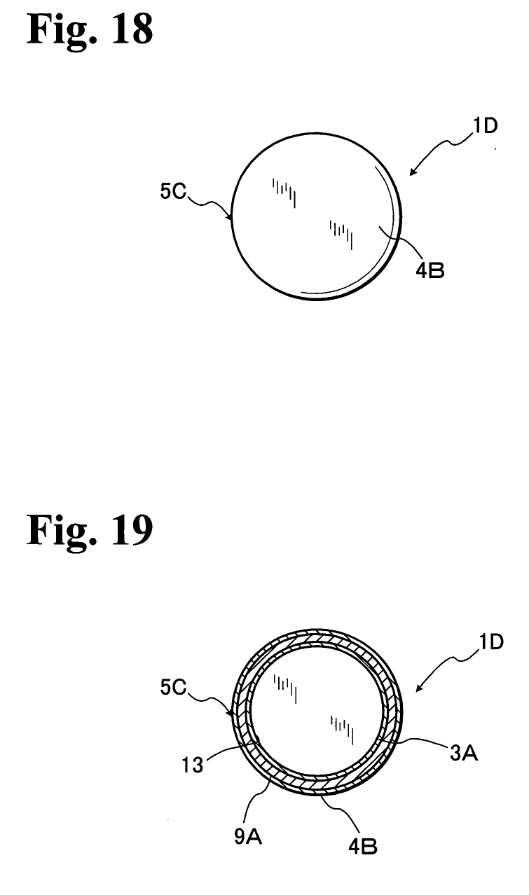

Other embodiments of the present invention will now be described referring to FIGS. 9 to 21. Through the drawings of the embodiments, like components are denoted by like numerals as of the first embodiment and will not be further explained in great detail.

A second embodiment of the present invention is shown in FIGS. 9 and 10 and is distinguished from the first embodiment by the fact that the urine absorbent body 5 is replaced from another urine absorbent body 5A which is formed in the shape of an ellipse. A urine absorbent article 1A in this way according to the second embodiment has similar advantages to that according to the first embodiment.

In this embodiment, the elliptical urine absorbent body 5 is explained, and the urine absorbent body 5 may be formed in the shape of a circle.

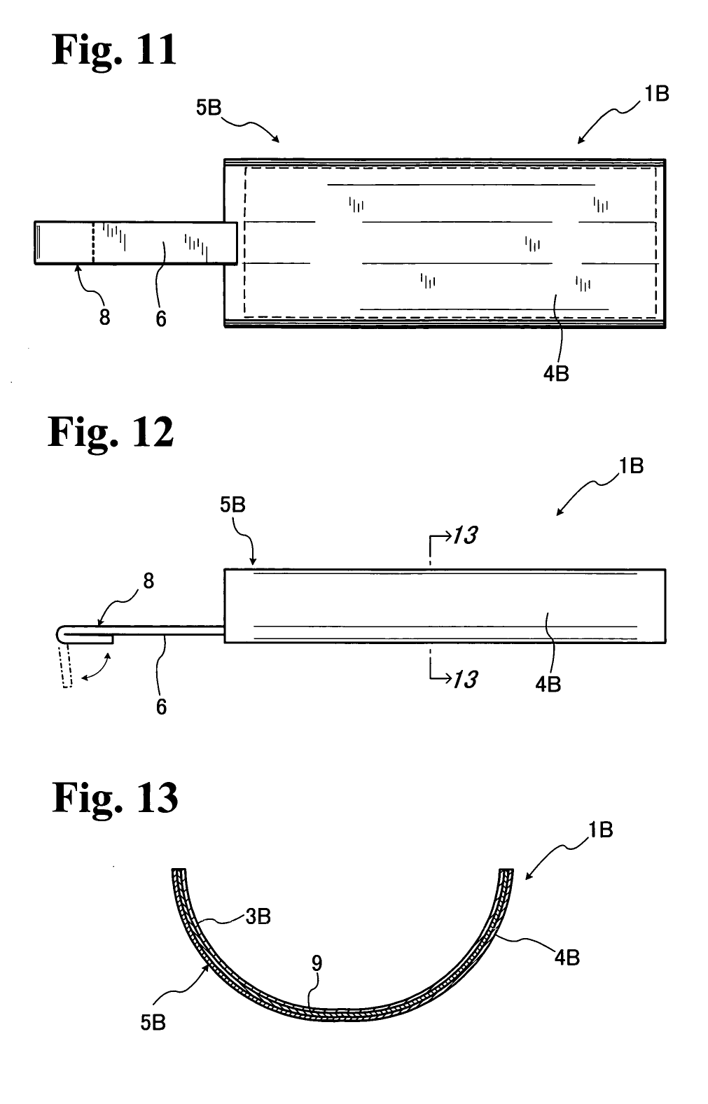

A third embodiment of the present invention is shown in FIGS. 11 to 13 and is distinguished from the first embodiment by the fact that the urine absorbent body 5 is replaced from another urine absorbent body 5B which is formed in the shape of an arc in section. A urine absorbent article 1B in this way according to the third embodiment has similar advantages to that according to the first embodiment.

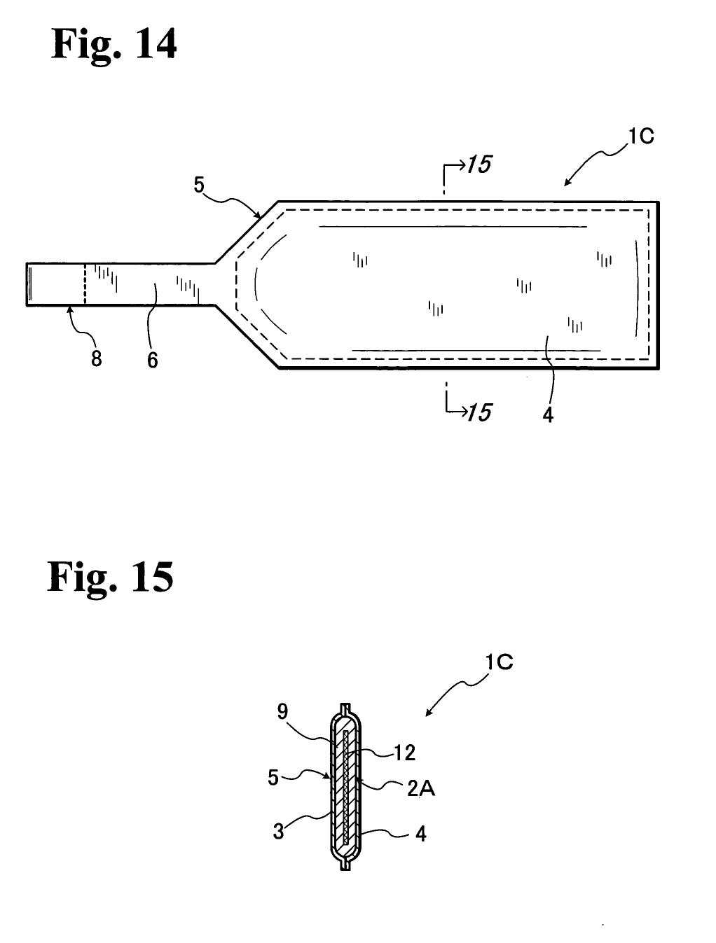

A fourth embodiment of the present invention is shown in FIGS. 14 and 15 and is distinguished from the first embodiment by the fact that the absorbent part 2 is replaced from another absorbent part 2A including an odor absorber 12 which can absorb the odor. A urine absorbent article 1C in this way according to the fourth embodiment has similar advantages to that according to the first embodiment. Therefore, when it leaves for a long time, and indoor environment can be kept good without filling with bad smell.

In addition, the odor absorber 12 may be wrapped in a water-soluble film, and it may be installed in the urine absorbent body 5.

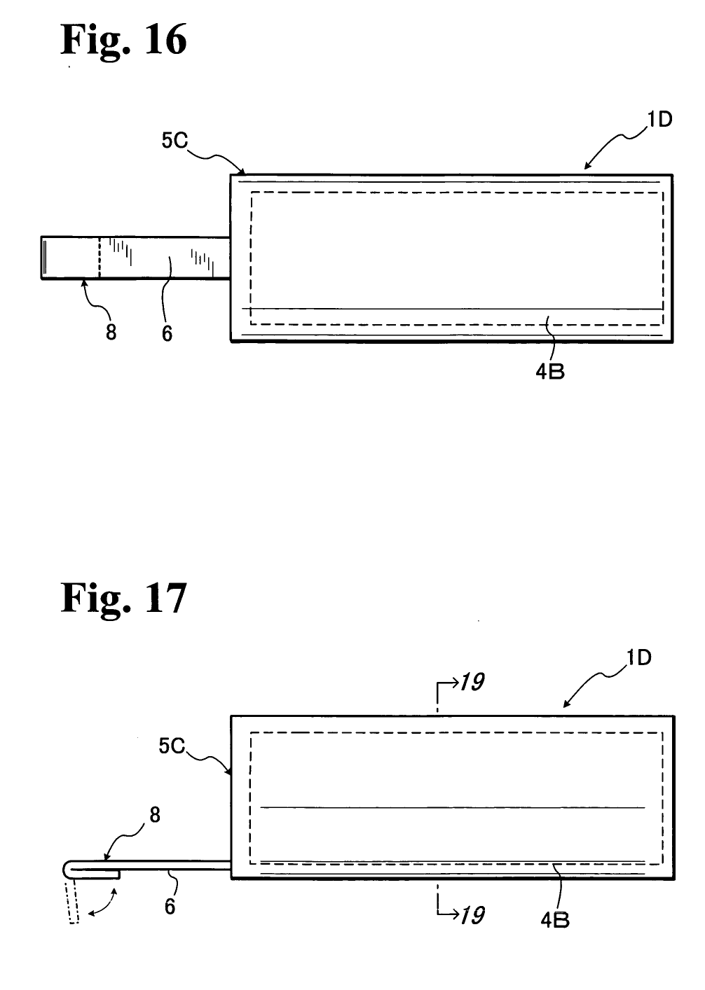

A fifth embodiment of the present invention is shown in FIGS. 16 to 19 and is distinguished from the first embodiment by the fact that the urine absorbent body 5 is replaced from another urine absorbent body 5C which is formed in the shape of a cylinder, having an opening 13 provided at a tip portion thereof. A urine absorbent article 1D in this way according to the fifth embodiment has similar advantages to that according to the first embodiment.

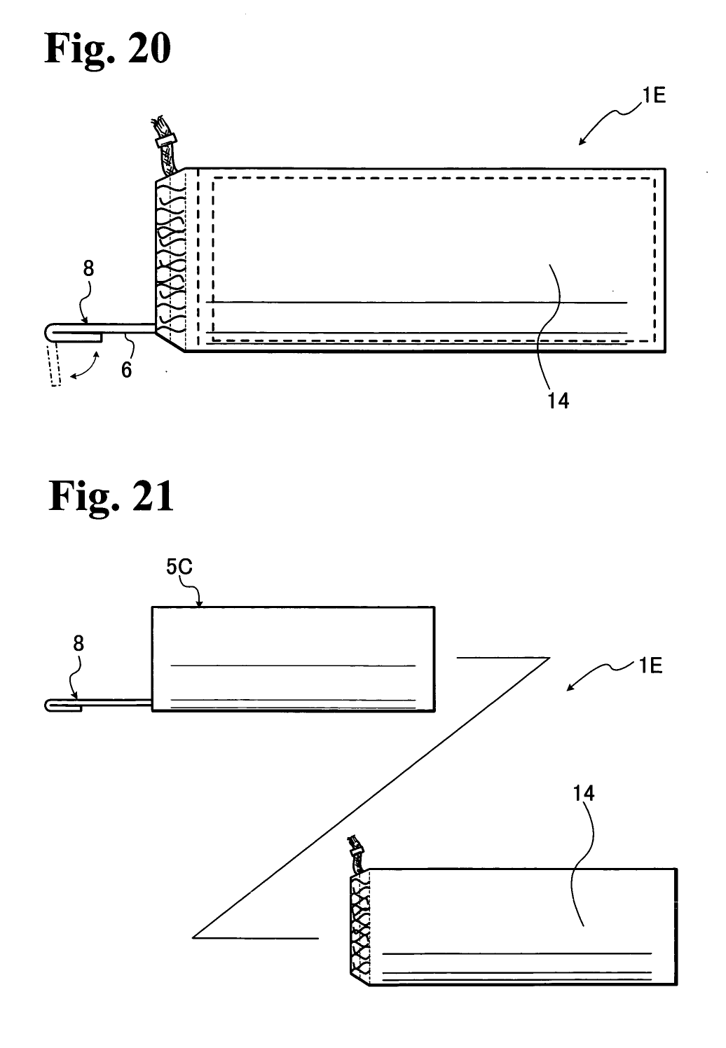

A sixth embodiment of the present invention is shown in FIGS. 20 and 21 and is distinguished from the fifth embodiment by the fact that a waterproof cover 14 is provided and is covered the outer circumferential part of the urine absorbent body 5C except the tip part thereof, capably of waterproofing. A urine absorbent article 1E in this way according to the sixth embodiment has similar advantages to that according to the fifth embodiment.

In addition, since the adhesive tape, fixation tape and the like may be attached to the outer part of the waterproof cover 14, capably of attaching to a bag, a container or the like, the urine absorbent article is stored into the container, and the users can bring when they go out and can always use it easily.

Furthermore, although each embodiment differs mainly explained based on the first embodiment, when the composition used for each embodiment uses the present invention is combined, the same effect is achieved.

As set forth above, the advantages of the invention are as follows:

(1) Since the urine absorbent article includes a urine absorbent body, having an absorbent part, capable of absorbing bodily fluids and moisture; a connection part which is attached to an end portion of the urine absorbent body; and a stopper which is attached to the connection part, capable of preventing to slip or come off from the urine absorbent body when in use, the sound of urination is muffled since it is absorbed by the absorbent part of the urine absorbent body quickly when the urine is discharged, and the urine does not stay in the urine bottle 11 since it is absorbed by absorbent part.

Therefore, since the urine is not spilt when the urine absorbent article is carried or dropped, it can be dealt with sanitarily without displeasure to other people.

(2) As discussed above, since the urine absorbent body with the absorbed urine is taken out and disposed, it can save the nurses and male nurse trouble.

(3) As discussed above, since it can manufacture cheaply, the user can use it without an economical burden.

(4) As discussed above, since the urine is absorbed in the urine absorbent body, indoor environment can be kept good without filling with bad smell when it is putting in the room for a long time.

(5) Claims 2 to 6 have similar advantages to the above-mentioned (1) to (4).

1. A urine absorbent article comprising:
a urine absorbent body, having an absorbent part including one of a cotton pulp, absorbable paper and high polymer absorber material, capable of absorbing bodily fluids and moisture;
a connection part which is attached to an end portion of the urine absorbent body; and
a stopper which is attached to the connection part, capable of preventing to slip or come off from the urine absorbent body when in use.

2. A urine absorbent article comprising:
a urine absorbent body, having an absorbent part including one of a cotton pulp, absorbable paper and high polymer absorber material, capable of absorbing bodily fluids and moisture;
a connection part which is attached to an end portion of the urine absorbent body; and
a stopper which is attached to the connection part, capable of preventing to slip or come off from the urine absorbent body when in use.
a waterproof cover, as a waterproof sheet, which is provided and covers the outer circumferential part of the urine absorbent body except the tip part thereof capably of waterproofing.

3. A urine absorbent article according to any one of claims 1 and 2, wherein the urine absorbent body is formed in the shape of a plain.

4. A urine absorbent article according to any one of claims 1 and 2, wherein the urine absorbent body is formed in the shape of one of a concave and arc.

5. A urine absorbent article according to any one of claims 1 and 2, wherein the urine absorbent body is formed in the shape of a tube.

6. A urine absorbent article according to claim 2, further comprising:
a fixation member including one of an adhesive tape and fixation tape, attaching the outer part of the waterproof cover, capably of attaching to a bag and a container.
